(19) United States
(12) Patent Application Publication
(10) Pub. No.: US 2013/0172625 A1
(43) Pub. Date: Jul. 4, 2013

(54) METHODS FOR USING ALLYLIC OXIDATION CATALYSTS TO PERFORM OXIDATION REACTIONS

(71) Applicants: Brian Tarbit, Ashington (GB); Graham J. Hutchings, Ross-On-Wye (GB); Jennifer K. Edwards, Cardiff (GB); Peter Miedziak, Cardiff (GB)

(72) Inventors: Brian Tarbit, Ashington (GB); Graham J. Hutchings, Ross-On-Wye (GB); Jennifer K. Edwards, Cardiff (GB); Peter Miedziak, Cardiff (GB)

(73) Assignee: VERTELLUS SPECIAL TIES INC

(21) Appl. No.: 13/581,068

(22) PCT Filed: Feb. 10, 2011
(86) PCT No.: PCT/US11/24326

§ 371 Date: Nov. 1, 2012

Related U.S. Application Data
(60) Provisional application No. 61/308,582, filed on Feb. 26, 2010.

(51) Int. Cl. C07C 45/34

(52) U.S. Cl. CPC C07C 45/34
USPC 568/344; 568/360; 568/374; 568/377; 422/208

(57) ABSTRACT

Methods for using ailylic oxidation catalysts to perform oxidation reactions. In an exemplary method for catalyzing an ailylic oxidation reaction of the present disclosure, the method comprises the step of catalyzing an oxidation of an ailylic compound using an ailylic oxidation catalyst. In at least one embodiment, the ailylic oxidation catalyst comprises palladium, gold, and titanium, In an exemplary embodiment, the ailylic oxidation catalyst comprises 2.5% Aυ÷2.5% Pd/TiO₂.

### PRIORITY

The present International Patent Application is related to, and claims the priority benefit of, U.S. Provisional Patent Application Ser. No. 61/308,582, filed Feb. 26, 2010, the contents of which are hereby incorporated by reference in their entirety into this disclosure.

### BACKGROUND

The disclosure of the present application introduces methods for using allylic oxidation catalysts to perform oxidation reactions. This disclosure references collaborative research performed at Cardiff University in Cardiff, United Kingdom, and at Vertellus Specialties UK Limited in Middlesbrough, England.

Allylic oxidation reactions, especially those performed in the fragrance and flavoring industries, are currently performed by using chrome-based and/or copper-based (heavy metal) oxidants as a catalyst. These heavy metal catalysts, including those resulting in residual Cr6+ (hexavalent chromium) from the oxidation reaction, are problematic if the end product has any sort of human use as hexavalent chromium it is a recognized carcinogen. This heavy metal catalyst problem has plagued the fragrance and flavoring industries for several decades as no alternatives to heavy metal catalysts have been known to exist.

A solution to this problem would be to identify one or more non-heavy metal catalysts useful for catalyzing allylic oxidation reactions, resulting in high conversion and selectivity rates and having no harmful reaction by-products. Thus, methods to perform such oxidation reactions using such catalysts would be well received in the chemical arts.

### BRIEF SUMMARY

In at least one embodiment of a method for catalyzing an allylic oxidation reaction of the present disclosure, the method comprises the step of catalyzing the oxidation of an allylic compound using an allylic oxidation catalyst. In another embodiment, the allylic oxidation catalyst comprises palladium, gold, and titanium. In yet another embodiment, the allylic oxidation catalyst comprises 2.5% Au+2.5% Pd/TiO₂.

In at least one embodiment of a method for catalyzing an allylic oxidation reaction of the present disclosure, the allylic oxidation catalyst comprises (a) gold and/or palladium, and (b) titanium, aluminum, silicon, iron, and/or carbon. In another embodiment, the allylic oxidation catalyst is free from chrome-based and copper-based oxidants. In yet another embodiment, the allylic oxidation catalyst comprises a catalyst selected from the group consisting of 1.0% Au+1.0% Pd/TiO₂, 1.0% Au+2.0% Pd/TiO₂, 3.0% Au+3.0% Pd/TiO₂, 4.0% Au+4.0% Pd/TiO₂, 5.0% Au+5.0% Pd/TiO₂, 2.0% Au+3.0% Pd/TiO₂, 3.0% Au+2.0% Pd/TiO₂, 1.0% Au+4.0% Pd/TiO₂, 4.0% Au+1.0% Pd/TiO₂, 2.0% Au+2.5% Pd/TiO₂, and 2.5% Au+2.0% Pd/TiO₂. In an additional embodiment, the allylic oxidation catalyst comprises a catalyst selected from the group consisting of 2.5% Au+2.5% Pd/Al₂O₃, 2.5% Au+2.5% Pd/SiO₂, 25% Au+2.5% Pd/Fe₂O₃, 2.5% Au+2.5% Pd/C, 2.5% Au/TiO₂, 2.5% Au/Al₂O₃, 2.5% Au/SiO₂, 2.5% Au/Fe₂O₃, 2.5% Au/C, 2.5% Pd/TiO₂, 2.5% Pd/Al₂O₃, 2.5% Pd/SiO₂, 2.5% Pd/Fe₂O₃, and 2.5% Pd/C.

In at least one embodiment of a method for catalyzing an allylic oxidation reaction of the present disclosure, the allylic compound comprises α-pinene, and the allylic oxidation catalyst catalyzes an oxidation of α-pinene to form at least one oxidized version of α-pinene. In an additional embodiment, the at least one oxidized version of α-pinene comprises verbenone. In yet an additional embodiment, the allylic compound comprises valencene, and the allylic oxidation catalyst catalyzes an oxidation of valencene to form at least one oxidized version of valencene. In another embodiment, the at least one oxidized version of valencene comprises nookatone.

In at least one embodiment of a method for catalyzing an allylic oxidation reaction of the present disclosure, the allylic compound comprises isophorone, and the allylic oxidation catalyst catalyzes an oxidation of isophorone to form at least one oxidized version of isophorone. In another embodiment, the at least one oxidized version of isophorone comprises 4-oxoisophorone. In yet another embodiment, the allylic compound comprises guaiene, and the ailylic oxidation catalyst catalyzes an oxidation of guaiene to form at least one oxidized version of guaiene. In an additional embodiment, the at least one oxidized version of guaiene comprises rotundone. In yet an additional embodiment, the allylic oxidation catalyst catalyzes an oxidation of the allylic compound to form at least one fragrance compound. In another embodiment, the allylic oxidation catalyst catalyzes an oxidation of the allylic compound to form at least one flavor compound.

In at least one embodiment of a method for catalyzing an allylic oxidation reaction of the present disclosure, the method comprises the steps of introducing an allylic compound into a reaction chamber, introducing an allylic oxidation catalyst into the reaction chamber, purging the reaction chamber with oxygen, and raising the temperature of the allylic compound and the allylic oxidation catalyst within the reaction chamber to facilitate the oxidation of the allylic compound using the allylic oxidation catalyst. In another embodiment, the reaction chamber comprises a stainless steel autoclave. In yet another embodiment, the allylic compound comprises a compound selected from the group consisting of α-pinene, valencene, isophorone, and guaiene. In an additional embodiment, the allylic oxidation catalyst comprises 2.5% Au+2.5% Pd/TiO₂.

In at least one embodiment of a method for catalyzing an allylic oxidation reaction of the present disclosure, the allylic oxidation catalyst comprises (a) gold and/or palladium, and (b) titanium, aluminum, silicon, iron, and/or carbon. In an additional embodiment, the allylic oxidation catalyst is free from chrome-based and copper-based oxidants. In yet an additional embodiment, the allylic oxidation catalyst comprises a catalyst selected from the group consisting of 1.0% Au+1.0% Pd/TiO₂, 1.0% Au+2.0% Pd/TiO₂, 3.0% Au+3.0% Pd/TiO₂, 4.0% Au+4.0% Pd/TiO₂, 5.0% Au+5.0% Pd/TiO₂, 2.0% Au+3.0% Pd/TiO₂, 3.0% Au+2.0% Pd/TiO₂, 1.0% Au+4.0% Pd/TiO₂, 4.0% Au+1.0% Pd/TiO₂, 2.0% Au+2.5% Pd/TiO₂, and 2.5% Au+2.0% Pd/TiO₂. In another embodiment, the allylic oxidation catalyst comprises a catalyst selected from the group consisting of 2.5% Au+2.5% Pd/Al₂O₃, 2.5% Au+2.5% Pd/SiO₂, 2.5% Au+2.5% Pd/Fe₂O₃, 2.5% Au+2.5% Pd/C, 2.5% Au/TiO₂, 2.5% Au/Al₂O₃, 2.5% Au/SiO₂, 2.5% Au/Fe₂O₃, 2.5% Au/C, 2.5% Pd/TiO₂, 2.5% Pd/Al₂O₃, 2.5% Pd/SiO₂, 2.5% Pd/Fe₂O₃, and 2.5% Pd/C.

In at least one embodiment of a method for catalyzing an allylic oxidation reaction of the present disclosure, the step of purging the reaction chamber with oxygen comprises purging the reaction chamber using oxygen to leave the reaction chamber at a desired elevated pressure. In another embodiment, the desired elevated pressure is selected from the group consisting of about 10 bar, about 20 bar, about 30 bar, between about 15 bar and about 25 bar, between about 25 bar and 35 bar, and greater than about 1 bar. In yet another embodiment, the step of raising the temperature of the allylic compound and the allylic oxidation catalyst within the reaction chamber comprises raising the temperature to a level selected from the group consisting of at least about 50° C., at least about 60° C., at least about 75° C., and between about 40° C. and about 95° C.

In at least one embodiment of a method for catalyzing an allylic oxidation reaction of the present disclosure, the method further comprises the step of stirring the allylic compound and the allylic oxidation catalyst within the reaction chamber prior to and/or during the step of raising the temperature of the allylic compound and the allylic oxidation catalyst within the reaction chamber to facilitate the oxidation of the allylic compound. In an additional embodiment, the step of stirring the allylic compound and the allylic oxidation catalyst comprises stirring the allylic compound and the allylic oxidation catalyst within the reaction chamber at a speed of 1500 r.p.m. In yet an additional embodiment, the step of stirring the allylic compound and the allylic oxidation catalyst comprises stirring the allylic compound and the allylic oxidation catalyst within the reaction chamber at a speed between 100 r.p.m. and 2500 r.p.m. in another embodiment, the method further comprises the step of cooling the temperature within the reaction chamber.

In at least one embodiment of a system for oxidizing allylic compounds of the present disclosure, the system comprises a reaction chamber for receiving at least one allylic compound and at least one allylic oxidation catalyst, a gas source operably coupled to the reaction chamber, the gas source operable to introduce a gas into the reaction chamber to increase pressure within the reaction chamber, a heating source in conductive communication with the reaction chamber, the heating source operable to provide heat to the reaction chamber to increase temperature within the reaction chamber, a stirrer for stirring contents within the reaction chamber, and an amount of an allylic oxidation catalyst placed within the reaction chamber, the allylic oxidation catalyst comprising (a) gold and/or palladium, and (h) titanium, aluminum, silicon, iron, and/or carbon, wherein the allylic oxidation catalyst catalyzes the oxidation of the allylic compound after the allylic compound is placed within the reaction chamber with the allylic oxidation catalyst, and wherein the oxidation of the allylic compound produces an oxidized allylic compound. In an additional embodiment, the reaction chamber comprises a stainless steel autoclave. In yet an additional embodiment, the gas source comprises, a source of oxygen, and the gas comprises oxygen. In another embodiment, the allylic oxidation catalyst comprises 2.5% Au+2.5% Pd/TiO₂. In yet another embodiment, the allylic oxidation catalyst comprises palladium, gold, and titanium, in at least one embodiment of a system for oxidizing allylic compounds of the present disclosure, the allylic oxidation catalyst is free from chronic-based and copper-based oxidants. In another embodiment, the allylic oxidation catalyst comprises a catalyst selected from the group consisting of 1.0% Au+1.0% Pd/TiO₂, 1.0% Au+2.0% Pd/TiO₂, 3.0% Au+3.0% Pd/TiO₂, 4.0% Au+4.0% Pd/TiO₂, 5.0% Au+5.0% Pd/TiO₂, 2.0% Au+3.0% Pd/TiO₂, 3.0% Au+2.0% Pd/TiO₂, 1.0% Au+4.0% Pd/TiO₂, 4.0% Au+1.0% Pd/TiO₂, 2.0% Au+2.5% Pd/TiO₂, and 2.5% Au+2.0% Pd/TiO₂. In yet another embodiment, the allylic oxidation catalyst comprises a catalyst selected from the group consisting of 2.5% Au+2.5% Pd/Al₂O₃, 2.5% Au+2.5% Pd/SiO₂, 2.5% Au+2.5% Pd/Fe₂O₃, 2.5% Au+2.5% Pd/C, 2.5% Au/TiO₂, 2.5% Au/Al₂O₃, 2.5% Au/SiO₂, 2.5% Au/Fe₂O₃, 2.5% Au/C, 2.5% Pd/TiO₂, 2.5% Pd/Al₂O₃, 2.5% Pd/SiO₂, 2.5% Pd/Fe₂O₃, and 2.5% Pd/C. In an additional embodiment, the allylic compound is selected from the group consisting of α-pinene, valencene, isophorone, and guaiene, and the oxidized allylic compound is selected from the group consisting of verbenone, nookatone, 4-oxoisophorone, and rotundone.

In at least one embodiment of a method for catalyzing an allylic oxidation reaction of the present disclosure, the method comprises the steps of providing an allylic oxidation catalyst within a reaction chamber, the allylic oxidation catalyst comprising (a) gold and/or palladium, and (b) titanium, aluminum, silicon, iron, and/or carbon, introducing an allylic compound into a reaction chamber, increasing the pressure within the reaction chamber, increasing the temperature within the reaction chamber, stirring contents within the reaction chamber, and cooling contents within the reaction chamber, wherein at least part of the cooled contents comprise an oxidized allylic compound.

The present disclosure further discloses an oxidized allylic compound, the oxidized allylic compound prepared by combining an allylic compound and an allylic oxidation catalyst comprising (a) gold and/or palladium, and (b) titanium, aluminum, silicon, iron, and/or carbon. In an exemplary embodiment, the allylic compound comprises α-pinene, and the oxidized allylic compound comprises verbenone. In another embodiment, the allylic compound comprises valencene, and the oxidized allylic compound comprises nookatone. In yet another embodiment, the allylic compound comprises isophorone, and the oxidized allylic compound comprises 4-oxoisophorone. In an additional embodiment, the allylic compound comprises guaiene, and the oxidized allylic compound comprises rotundone

### BRIEF DESCRIPTION OF THE DRAWINGS

FIG. 1 shows graphical conversion results from various catalysts used to catalyze the reaction of α-pinene to form verbenone in accordance with the present disclosure;

FIG. 2 shows graphical selectivity results from various catalysts used to catalyze the reaction of α-pinene to form verbenone in accordance with the present disclosure;

FIG. 3 shows graphical conversion results at various pressures with and without the use of an allylic oxidation catalyst in accordance with the present disclosure;

FIG. 4 shows graphical selectivity results at various at various pressures with and without the use of an allylic oxidation catalyst in accordance with the present disclosure;

FIGS. 5A, 5B, and 5C show graphical conversion results of the oxidation of α-pinene over time at various pressures, said reaction catalyzed using an allylic oxidation catalyst in accordance with the present disclosure;

FIGS. 6A, 6B, and 6C show graphical selectivity results of the oxidation of n-pinene over time to form verbenone at various pressures, said reaction catalyzed using an allylic oxidation catalyst in accordance with the present disclosure;

FIG. 7 shows graphical conversion results of the oxidation of valencene over time, said reaction catalyzed using an allylic oxidation catalyst in accordance with the present disclosure;

FIG. 8 shows graphical selectivity results of the oxidation of valencene over time to form nookatone, said reaction catalyzed using an allylic oxidation catalyst in accordance with the present disclosure;

FIG. 9 shows graphical conversion results of the oxidation of isophorone over time, said reaction catalyzed using an allylic oxidation catalyst in accordance with the present disclosure; and

FIG. 10 shows graphical selectivity results of the oxidation of isophorone over time to form 4-oxoisophorone, said reaction catalyzed using an allylic oxidation catalyst in accordance with the present disclosure.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The disclosure of the present application discloses methods for performing allylic oxidation reactions using various allylic oxidation catalysts. In at least one embodiment, an allylic oxidation catalyst comprising gold, palladium, and titanium is useful to perform said allylic oxidation reactions. Exemplary allylic oxidation reactions include, but are not limited to, the oxidation of α-pinene to form verbenone, the oxidation of valencene to form nookatone, and the oxidation of isophorone to form 4-oxoisophorone. The disclosure of the present application is not intended to be limited to the three aforementioned reactions, as various other allylic oxidation reactions are contemplated using one or more of the allylic oxidation catalysts referenced herein.

En at least one embodiment of an allylic oxidation reaction of the disclosure of the present application, the allylic oxidation catalyst is used to catalyze the reaction of the oxidation of α-pinene ((1S,5S)-2,6,6-Trimethylbicyclo[3.1.1]hept-2-ene((-)-α-Pinene))) to form verbenone ((1R)-cis-4,6,6-Trimethylbicyclo-[3.1.1]hept-3-en-2-one) as shown in Reaction No. 1 below:

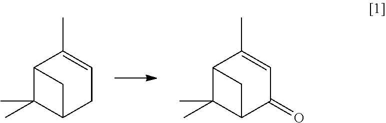

In at least one example, the above reaction was performed using an allylic oxidation catalyst comprising 2.5% Au+2.5% Pd/TiO₂, resulting in an essentially total conversion of α-pinene with greater than 90% oxidation selectivity. This level of selectivity has been previously unknown in the chemical arts for this particular reaction. Such a reaction, as referenced in the Background of the present application, is typically currently performed by using chrome-based and/or copper-based (heavy metal) oxidants as a catalyst, noting that residual Cr6+ (hexavalent chromium) is a concern if the product has any sort of human use because hexavalent chromium is a recognized carcinogen.

In at least an additional embodiment of an allylic oxidation reaction of the disclosure of the present application, an allylic oxidation catalyst is used to catalyze the reaction of the oxidation of valencene ((1R,7R,8αS)-1,2,3,5,6,7,8,8a-Octahydro-1,8a-dimethyl-7-(1-methylethenyl)naphthalene) to form nookatorte (4-alpha,5-dimethyl-1,2,3,4,4alpha,5,6,7-octahydro-7-keto-3-isopropenylnaphthalene) as shown in Reaction No. 2 below:

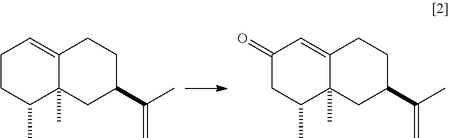

This particular reaction results in about 70% conversion, which is relatively high for a non-chromium catalyzed reaction. In addition, this reaction is very selective when performed using an allylic oxidation catalyst of the disclosure of the present application, which is not expected when this reaction is performed using other catalysts known in the art.

In at least another embodiment of an allylic oxidation reaction of the disclosure of the present application, an allylic oxidation catalyst is used to catalyze the reaction of the oxidation of isophorone (3,5,5-Trimethyl-2-cyclohexen-1-one) to form 4-oxoisophorone (2,6,6-Trimethyl-2-cyclohexene-1,4-dione) as shown in Reaction No. 3 below;

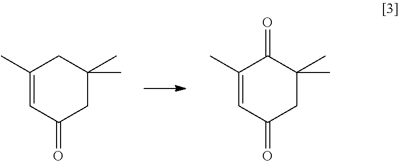

In yet another embodiment of an allylic oxidation reaction of the disclosure of the present application, an allylic oxidation catalyst is used to catalyze the reaction of the oxidation of guaiene ((1S-cis)-1,2,3,4,5,6,7,8-octahydro-7-isopropylidene-1,4-dimethylazulene) to form rotundone as shown in Reaction No. 4 below:

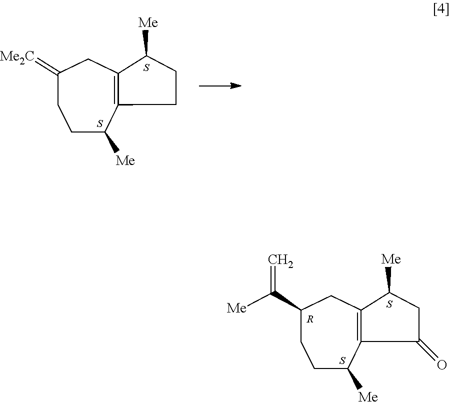

An exemplary catalyst used to perform the above-referenced reactions (2.5% Au+2.5% Pd/TiO₂) was been prepared by dissolving palladium chloride in HAuCl₄, adding that resultant solution to titanium and then drying, grinding, and calcining the resulting powder to obtain the catalyst used in the reaction.

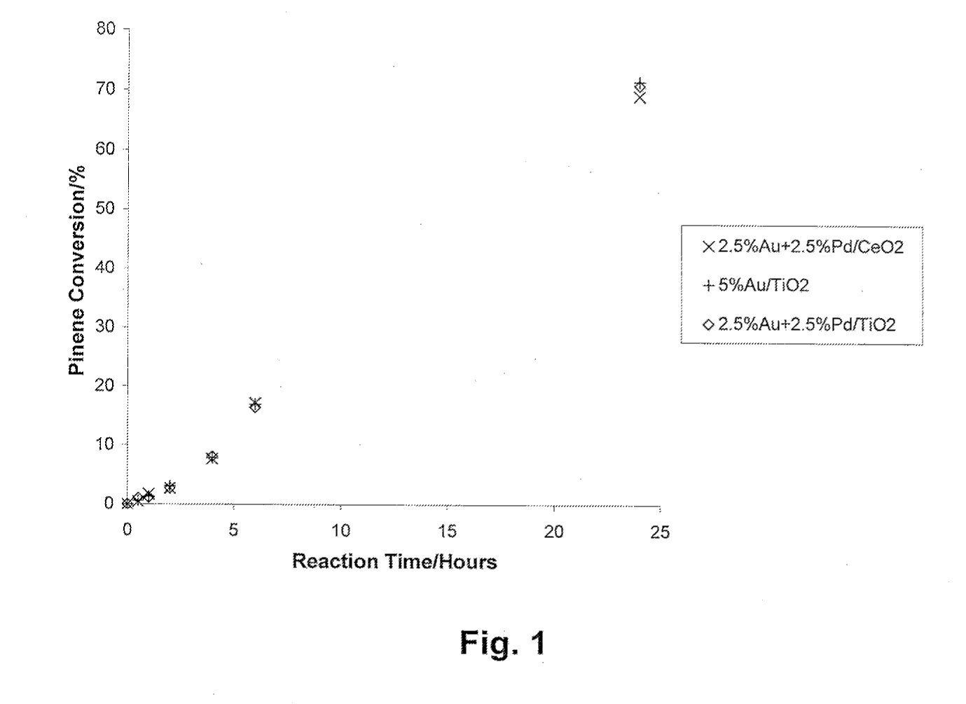

To determine the effect of potential catalysts on catalyzing the reaction of α-pinene to form verbenone (Reaction No. 1 referenced herein), several catalyst candidates were tested, including 2.5% Au+2.5% Pd/TiO₂, 2.5% Au+2.5% Pd/CeO₂, and 5% Au/TiO₂. In one experiment, these catalysts were used at a pressure of 10 bar in attempt to oxidize α-pinene, with overall conversion results shown in FIG. 1. As shown in FIG. 1, each catalyst resulted in approximately the same percentage of conversion of α-pinene to an oxidized farm of the compound, showing no real statistical difference between any of the aforementioned tested catalysts.

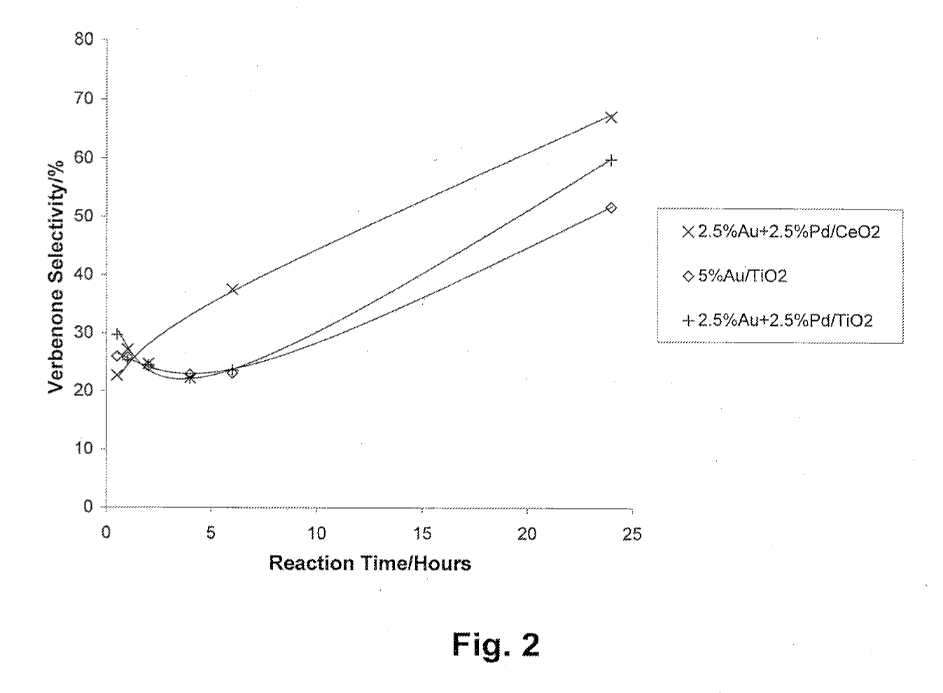

FIG. 2 shows graphical conversion results from various catalysts used to catalyze the oxidization reaction of α-pinene in attempt to specifically form verbenone. As shown in FIG. 2, catalysts containing gold (Au) and palladium (Pd) resulted in higher reaction selectivity after about 24 hours as compared to catalysts without palladium. This result was unexpected as each tested catalyst resulted in approximately the same percentage of conversion of α-pinene (as shown in FIG. 1) to an oxidized product, but depending on the type of catalyst used, the overall selectivity of the allylic oxidation of α-pinene to form verbenone was improved.

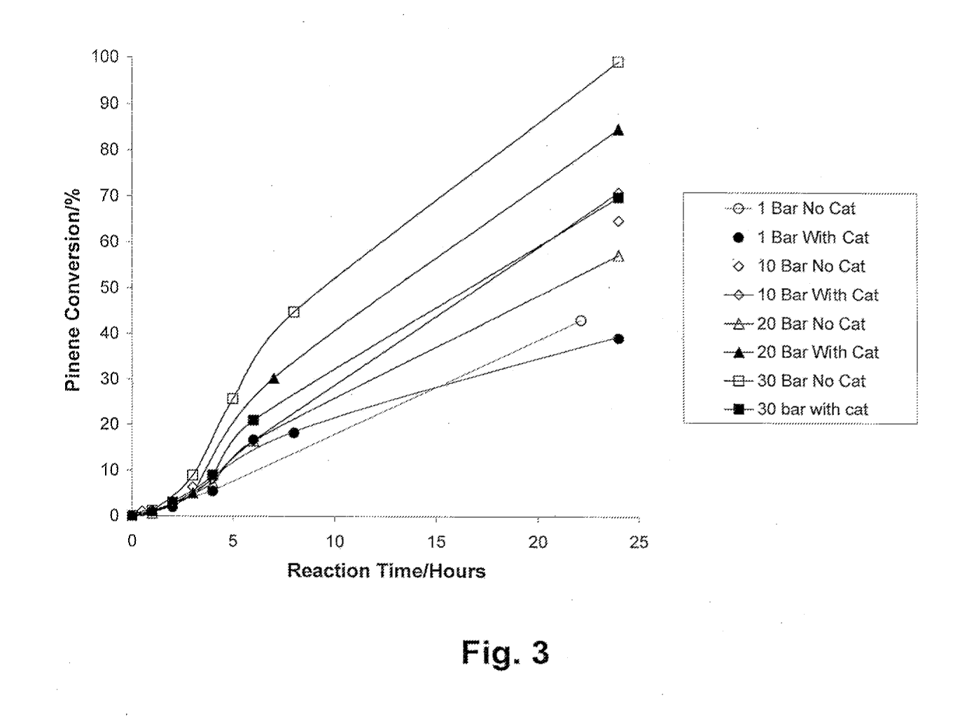

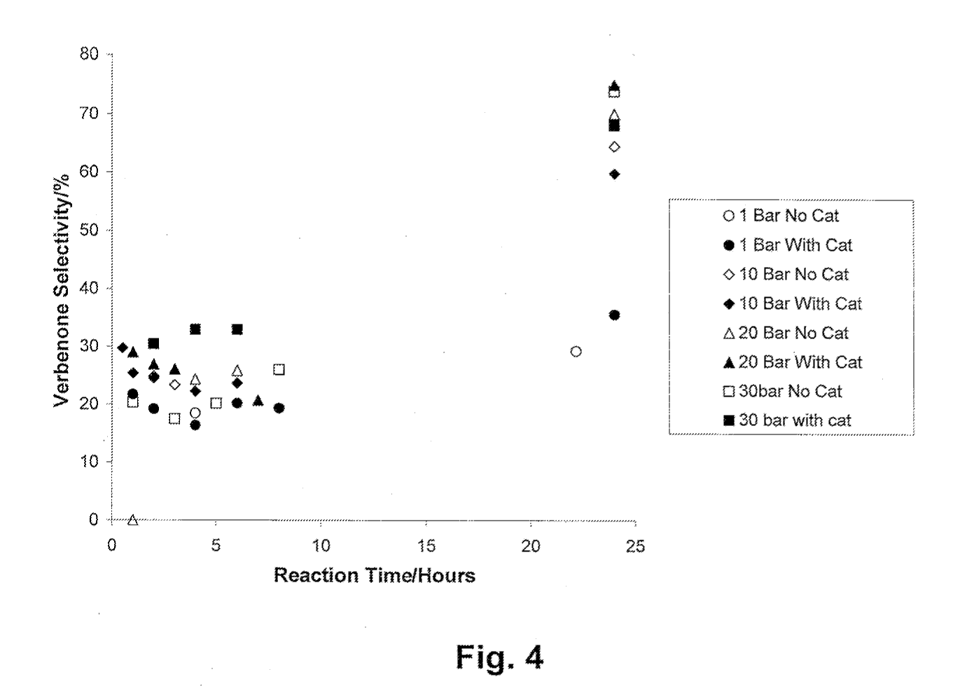

Exemplary allylic oxidation reactions to oxidize α-pinene were also performed at various pressures to determine the effect of pressure on reaction conversion and selectivity rates. FIG. 3 shows graphical conversion results at various pressures with and without the use of an allylic oxidation catalyst. As shown in FIG. 3, pressure has a significant effect on the oxidation reaction, noting that at higher pressures, the difference between the overall conversion with and without the use of an allylic oxidation catalysts is more pronounced. FIG. 4 shows graphical selectivity results at various at various pressures with and without the use of an allylic oxidation catalyst.

As shown in FIG. 4, pressure has a significant effect on the overall selectivity of the oxidation reaction, noting that higher selectivity rates exist at higher pressures. For the reactions shown in FIGS. 3 and 4, catalyst testing was performed using a stainless steel autoclave charged with α-pinene (Fluka, 40 ml) and an allylic oxidation catalyst, maintaining the reaction using a stirrer speed of 1500 r.p.m., with the reaction mixture raised and maintained at the desired reaction temperature (75° C.) for the desired amount of time (24 h).

The disclosure of the present application emphasizes the use of non-heavy metals (gold and palladium, for example), instead of chromium and copper, as catalyst components for oxidation reactions. Although one particular gold and palladium catalyst (2.5% Au+2.5% Pd/TiO₂) is referenced throughout the present application, exemplary allylic oxidation catalysts, and exemplary oxidation reactions using said allylic oxidation catalysts, are not limited to the use of a single gold and palladium catalyst. Additional exemplary catalysts include, but are not limited to, 1.0% Au+1.0% Pd/TiO₂, 1.0% Au+2.0% Pd/TiO₂, 3.0% Au+3.0% Pd/TiO₂, 4.0% Au+4.0% Pd/TiO₂, 5.0% Au+5.0% Pd/TiO₂, 2.0% Au+3.0% Pd/TiO₂, 3.0% Au+2.0% Pd/TiO₂, 1.0% Au+4.0% Pd/TiO₂, 4.0% Au+1.0% Pd/TiO₂, 2.0% Au+2.5% Pd/TiO₂, 2.5% Au+2.0% Pd/TiO₂, and catalysts containing higher or lower amounts of gold and/or palladium other than those recited in this non-exhaustive list.

In addition to the foregoing, the disclosure of the present application extends to allylic oxidation catalysts other than those comprising gold and palladium along with titanium. For example, allylic oxidation catalysts comprising gold and palladium along with aluminum (from, for example. Al₂O₃), silicon (from, for example. SiO₂), iron (from, for example, Fe₂O₃), and carbon may be used to catalyze allylic oxidation reactions as disclosed herein. In addition, exemplary catalysts may comprise gold or palladium along with titanium (from TiO₂), aluminum, silicon, iron, and/or carbon as disclosed herein. Additional exemplary catalysts include, but are not limited to, 2.5% Au+2.5% Pd/Al₂O₃, 2.5% Au+2.5% Pd/SiO₂, 2.5% Au+2.5% Pd/Fe₂O₃, 2.5% Au+2.5% Pd/C, 2.5% Au/TiO₂, 2.5% Au/Al₂O₃, 2.5% Au/SiO₂, 2.5% Au/Fe₂O₃, 2.5% Au/C, 2.5% Pd/TiO₂, 2.5% Pd/Al₂O₃, 2.5% Pd/SiO₂, 2.5% Pd/Fe₂O₃, and 2.5% Pd/C, and catalysts containing higher or lower amounts of gold and/or palladium other than those recited in this non-exhaustive list.

### Example 1

### Oxidation of α-pinene to Form Verbenone

In an exemplary α-pinene oxidation experiment, catalyst testing was performed using a Parr Instruments stainless steel autoclave with a nominal volume of 50 ml and a maximum working pressure of 3000 Psi. The reactor was charged with α-pinene (Fluka, 20 ml) and catalyst (2.5% Au+2.5% Pd/TiO₂, 50 mg). The autoclave was then purged 3 times with oxygen leaving the vessel at the desired pressure (30 bar). The pressure was maintained constant throughout the experiment, and as the oxygen was consumed in the reaction it was replenished. The stirrer speed was set at 1500 r.p.m. and the reaction mixture was raised and maintained at the desired reaction temperature (75° C.) for the desired amount of time (28 h). The desired reaction temperature (75° C. was not randomly chosen, noting that test reactions at 50° C. demonstrated relatively slow conversion (approximately 30% after 52 h), and reactions at 100° C. were successfully carried out, but reaction runaway occurred making sample collection and testing difficult. Samples were taken from the final reaction mixture and analyzed by GC using a CP-Wax column. The reaction was originally carried out over a period of 24 h and near total conversion was achieved; however, for the last testing batch, the reaction time was extended to 28 h to ensure almost complete conversion. The reaction was also carried out in the absence of catalyst, wherein after 24 h a conversion of about 80% would be expected.

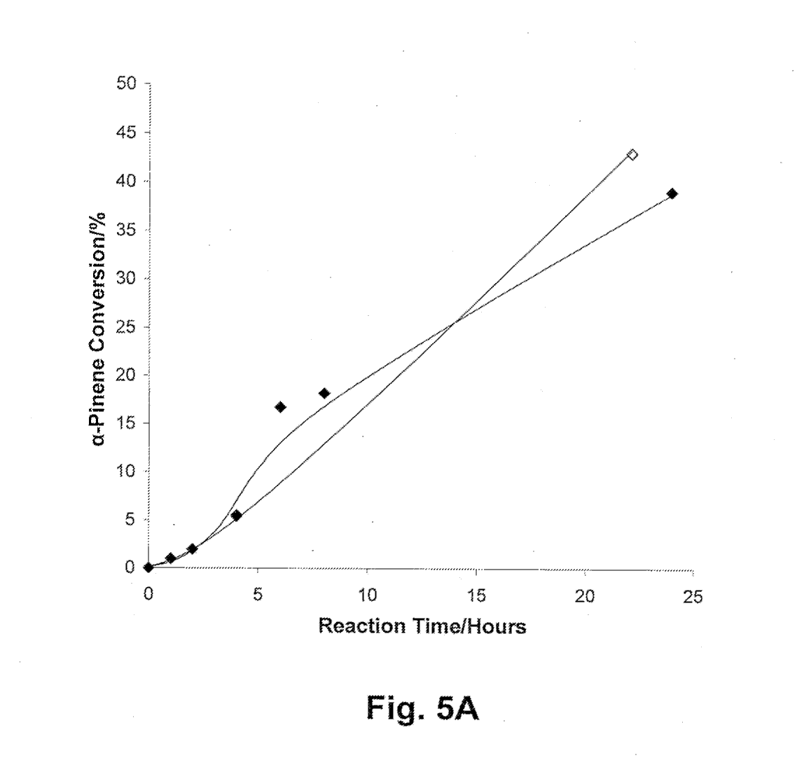

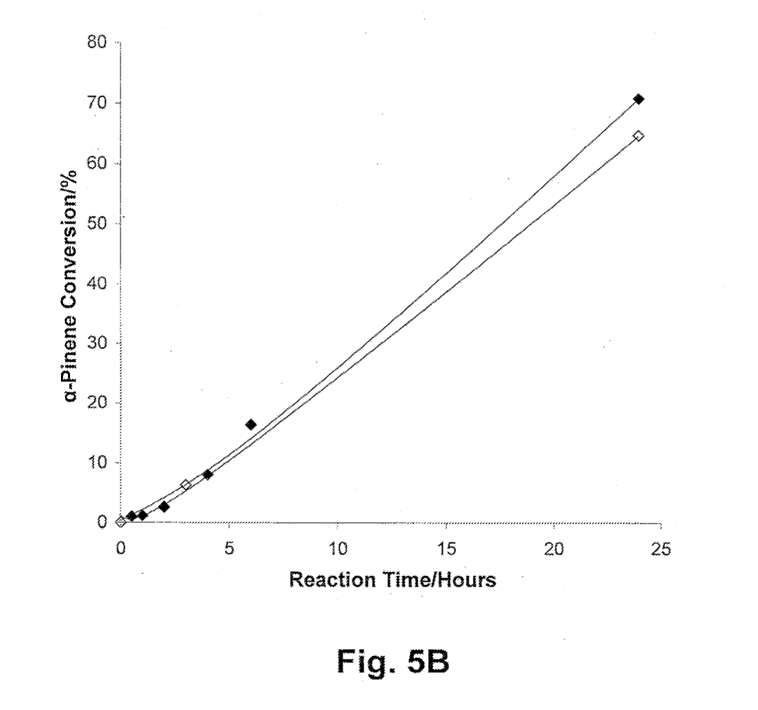

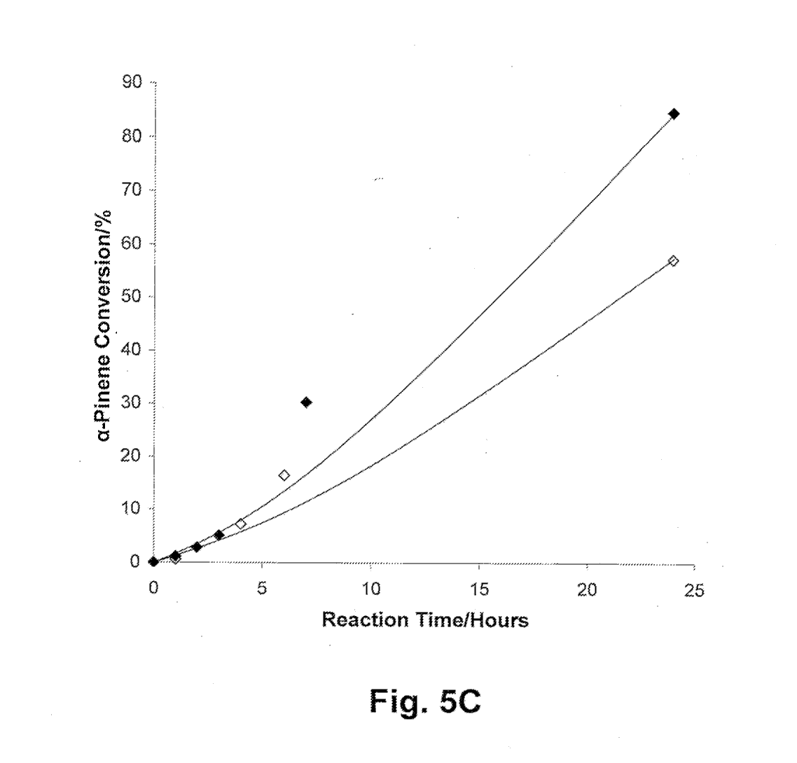

FIGS. 5A, 5B, and 5C show the percentage of conversion of α-pinene to an oxidized product over time and under differing pressure conditions based upon samples tested in accordance with Example 1. As shown in FIG. 5A, conversion without catalyst (open shape) was slower earlier on, but at approximately 24 h, conversion without catalyst was slightly higher at 1 bar. The shaded shapes represent the use of catalyst of the present disclosure. FIG. 5B shows conversion results at 10 bar, whereby total percent conversion to α-pinene was higher at 24 h using a catalyst of the present disclosure (shaded) than without the use of a catalyst (unshaded). FIG. 5C shows reaction results at 20 bar, whereby the use of an exemplary catalyst of the present disclosure (shaded) has a significantly higher percentage of conversion to α-pinene than without the use of a catalyst (unshaded), especially at 24 h.

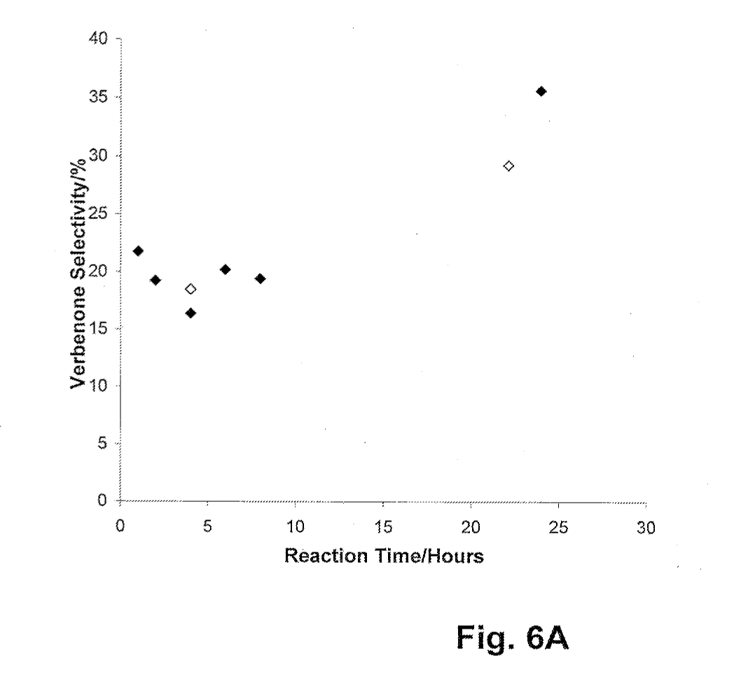

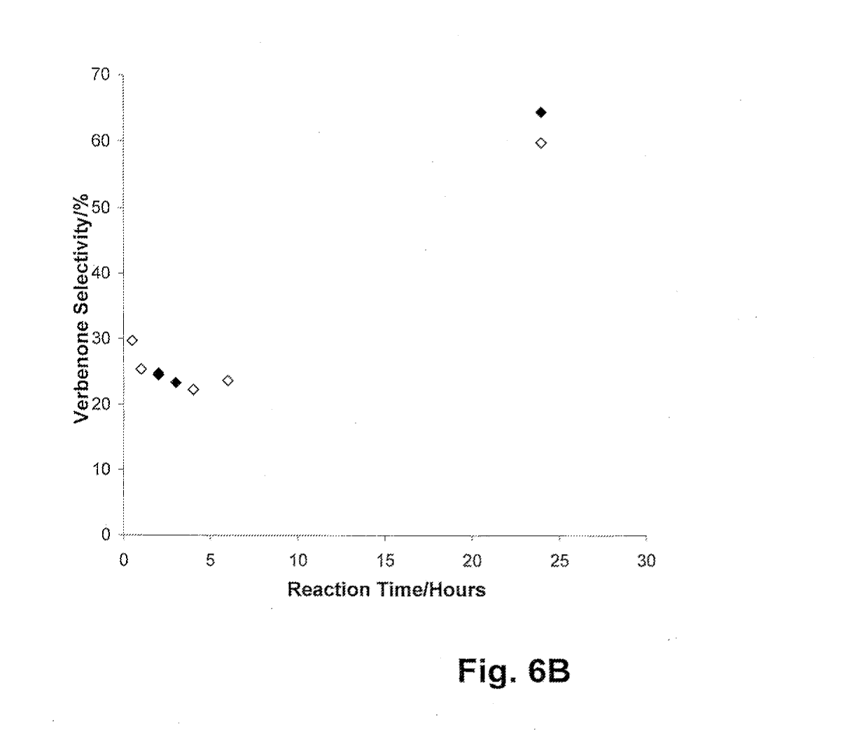

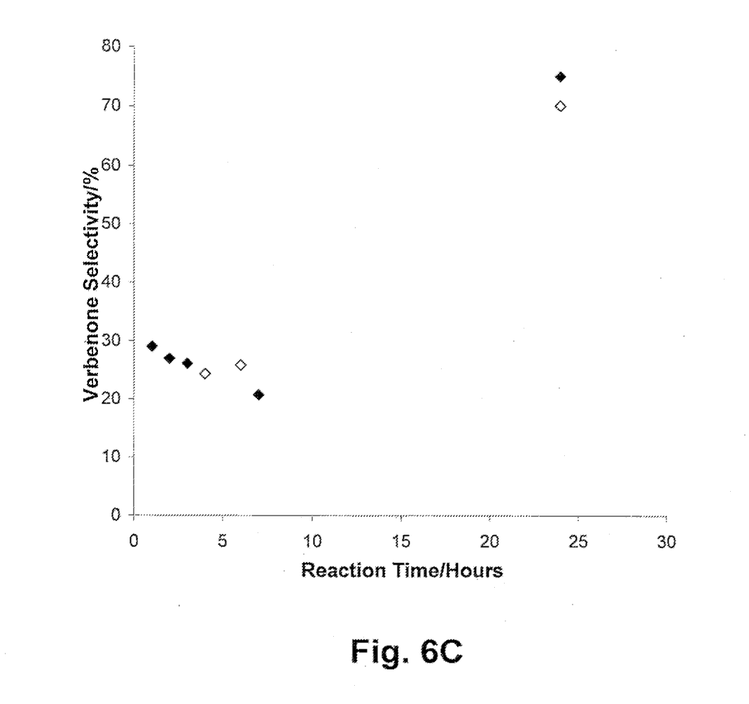

FIGS. 6A, 6B, and 6C show data pertaining to the overall selectivity of the α-pinene oxidation reaction in accordance with Example 1. As shown in FIG. 6A, the overall selectivity of verbenone as the reaction product of the oxidation of α-pinene was significantly higher at 24 h when using a catalyst (shaded shapes) as compared to no catalyst (unshaded). FIG. 6B shows reaction data at 10 bar, and FIG. 6C shows reaction data at 20 bar, noting that in each example, verbenone selectivity was higher in the presence of a catalyst (shaded) at 24 h.

For the above-referenced experiment, the catalyst (2.5% Au+2.5% Pd/TiO₂) was prepared in accordance with the following procedure. Palladium chloride (Johnson Matthey, 83.3 mg) was dissolved in a stirred and heated aqueous solution (5 ml) of HAuCl₄ (Johnson Matthey, 5 g in 250 ml water solution). The resultant solution was added to the titanium (Degussa, 1.9 g) and the resulting slurry was dried at 120° C. for 16 h. The resulting powder was ground and calcined (1 g, 6 inch quartz boat) in static air at 40° C. for 3 hours at a ramp rate of 20° C./min.

### Example 2

### Oxidation of Valencene to Form Nookatone

In an exemplary valencene oxidation experiment, catalyst testing was performed using an Autoclave Engineers stainless steel autoclave (Autoclave Engineers Inline MagneDrive III) with a nominal volume of 100 ml and a maximum working pressure of 2000 psi. The vessel was charged with valencene (40 ml) and catalyst (50 mg of 2.5 wt % Au+2.5 wt % Pd/TiO₂, prepared by a deposition precipitation method). In this example, Palladium Nitrate (119 mg) was dissolved in a stirred and heated aqueous solution (5 ml) of HAuCl₄ (5 g in 250 ml water) and this solution was added to a stirred, heated (60° C.) slurry of titanium (P25 degussa) (1.9 g) in water (300 ml). Sodium carbonate (1M) was added drop wise until the solution reached pH 8. The solution was maintained at pH 8 for 1 hour. The slurry was then filtered, washed with de-mineralized water (1 L). The washed solid was dried at 80° C. for 16 h. The resulting powder was ground and calcined (1 g, 6 inch quartz boat) in static air at 400° C. for 3 hours at a ramp rate of 20° C. min⁻¹.

The autoclave was then purged three times with oxygen, leaving the vessel at the desired pressure (30 bar). The pressure was maintained constant throughout the experiment; as the oxygen was consumed in the reaction it was replenished. The stirrer speed was set at 1500 r.p.m. and the reaction mixture was raised and maintained at the desired reaction temperature of 80° C. for 72 h. Samples from the reactor were taken periodically using a sampling pipe, ensuring that the volume purged before sampling was higher than the tube volume, and the extracted samples were analyzed by gas chromatography (GC) using a CP-Wax column.

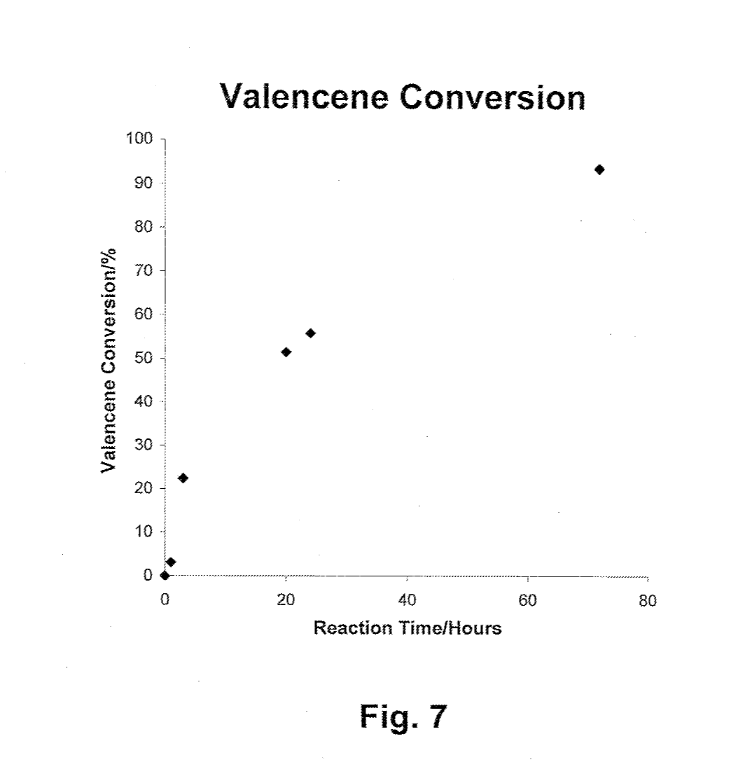

FIG. 7 shows the percentage of conversion of valencene to an oxidized product over time based upon samples tested in accordance with Example 2. As shown in FIG. 7, the conversion rate was highest at the start of the reaction, and after approximately 72 hours, the overall conversion of valencene exceeded 90%.

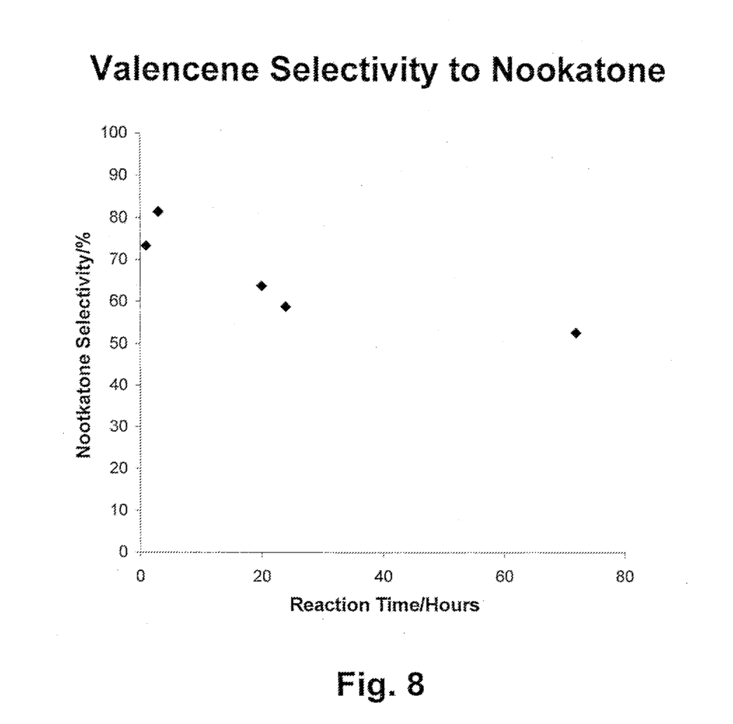

FIG. 8 shows data pertaining to the overall selectivity of the valencene oxidation reaction in accordance with Example 2. As shown in FIG. 8, the overall selectivity of nookatone as the reaction product of the oxidation of valencene was initially in the 70-80% range, tapering off to approximately 50% after approximately 72 hours of reaction time.

### Example 3

### Oxidation of Isophorone to Form 4-oxoisophorone

In an exemplary isophorone oxidation experiment, catalyst testing was performed using an Autoclave Engineers stainless steel autoclave (Autoclave Engineers Inline MagneDrive III) with a nominal volume of 100 ml and a maximum working pressure of 2000 psi. The vessel was charged with isophorone (40 ml) and catalyst (50 mg of 2.5 wt % Au+2.5 wt % Pd/TiO₂, prepared by the deposition precipitation method as referenced herein. The autoclave was then purged 3 times with oxygen leaving the vessel at the desired pressure (10 bar). The pressure was maintained constant throughout the experiment, and as the oxygen was consumed in the reaction it was replenished. The stirrer speed was set at 1500 r.p.m. and the reaction mixture was raised and maintained at the desired reaction temperature (75° C.) for 24 h. Samples from the reactor were taken periodically using a sampling pipe, ensuring that the volume purged before sampling was higher than the tube volume. The samples were then analyzed by GC using a CP-Wax column.

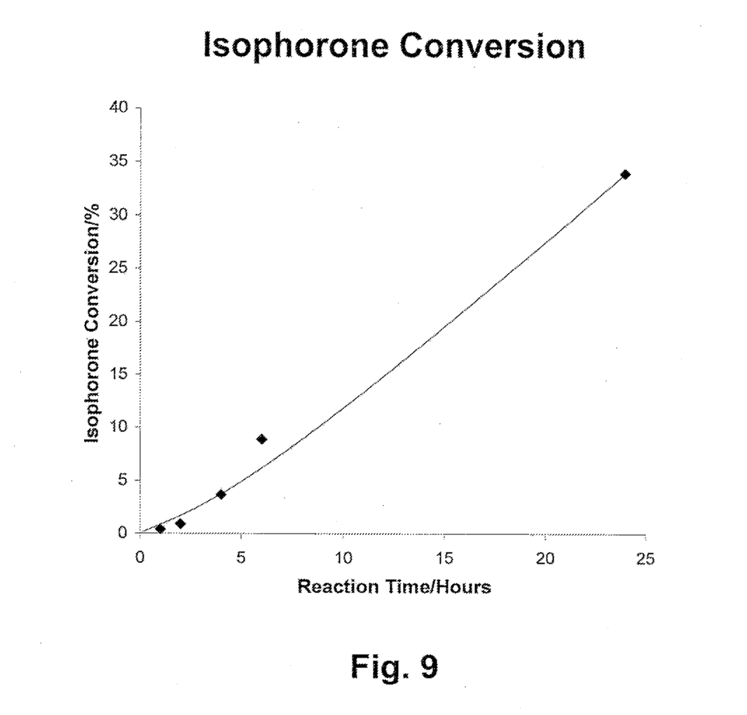

FIG. 9 shows the percentage of conversion of isophorone to an oxidized product over time based upon samples tested in accordance with Example 3. As shown in FIG. 9, approximately 30-35% of isophorone was converted to an oxidized product after 24 h, with a higher rate of oxidation occurring within the first few hours of the reaction.

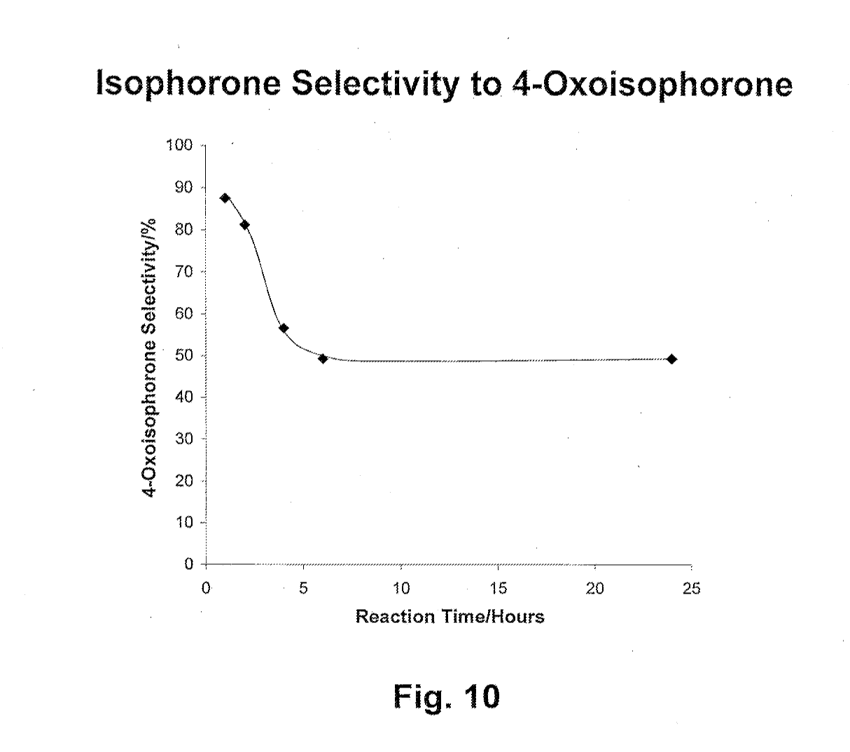

FIG. 10 shows data pertaining to the overall selectivity of the isophorone oxidation reaction in accordance with Example 3. As shown in FIG. 10, the overall selectivity of 4-oxoisophorone as the reaction product of the oxidation of isophorone appears to be the initially formed product, with other products forming as the reaction progresses, noting the leveling out to a selectivity of 4-oxoisophorone of about 50%.

### Example 4

### Oxidation of Guaiene to Form Rotundone

In an exemplary guaiene oxidation experiment, catalyst testing was performed using a Parr Instruments stainless steel autoclave with a nominal volume of 100 ml and a maximum working pressure of 873 psi. The reactor was charged with guaiene (0.22 mmol, 40 ml) and catalyst (2.5% Au 2.5% Pd/TiO₂, 50 mg). The autoclave was then purged 5 times with oxygen leaving the vessel at the desired pressure (30 bar). The pressure was maintained constant throughout the experiment, and as the oxygen was consumed in the reaction it was replenished. The stirrer speed was set at 1500 r.p.m. and the reaction mixture was raised and maintained at the desired reaction temperature (80° C.) for the desired amount of time (30 h). Visual inspection of the samples indicated that the oxidation reaction had taken place.

The Examples provided above are merely exemplary examples of dyke oxidation reactions of the disclosure of the present application and the conditions by which the exemplary reactions were performed. It is well within the scope of the present disclosure to modify one or more reaction parameters, including, but not limited to, modifying the elevated reaction pressure (greater than 1 bar, 5-15 bar, 20-25 bar, 30-35 bar, 10 bar, 20 bar, 30 bar, etc.), modifying the elevated reaction temperature (at least 50° C., at least 60° C., at least 75° C., at least 80° C., modifying the stirring speed (between 100 r.p.m., 1500 r.p.m., 2500 r.p.m., etc), and/or modifying the amounts of reactants and/or catalysts.

While various embodiments of allylic oxidation catalysts and methods for using the same to perform allylic oxidation reactions have been described in considerable detail herein, the embodiments are merely offered by way of non-limiting examples of the disclosure described herein. It will therefore be understood that various changes and modifications may be made, and equivalents may be substituted for elements thereof, without departing from the scope of the disclosure. Indeed, this disclosure is not intended to be exhaustive or to limit the scope of the disclosure.

Further, in describing representative embodiments, the disclosure may have presented a method and/or process as a particular sequence of steps. However, to the extent that the method or process does not rely on the particular order of steps set forth herein, the method or process should not be limited to the particular sequence of steps described. Other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1-47. (canceled)

48. A method for catalyzing an allylic oxidation reaction, the method comprising the step of catalyzing an oxidation of an allylic compound using an allylic oxidation catalyst wherein the allylic oxidation catalyst comprises a catalyst selected from the group consisting of 2.5% Au+2.5% Pd/TiO₂, 1.00% Au+1.0% Pd/TiO₂, 1.0% Au+2.0% Pd/TiO₂, 3.0% Au+3.0% Pd/TiO₂, 4.0% Au+4.0% Pd/TiO₂, 5.0% Au+5.0% Pd/TiO₂, 2.0% Au+3.0% Pd/TiO₂, 3.0% Au+2.0% Pd/TiO₂, 1.0% Au+4.0% Pd/TiO₂, 4.0% Au+1.0% Pd/TiO₂, 2.0% Au+2.5% Pd/TiO₂, 2.5% Au+2% Pd/TiO₂, 2.5% Au+2.5% Pd/Al₂O₃, 2.5% Au+2.5% Pd/SiO₂, 2.5% Au+2.5% Pd/Fe₂O₃, 2.5% Au+2.5% Pd/C, 2.5% Au/TiO₂, 2.5% Au/Al₂O₃, 2.5% Au/SiO₂, 2.5% Au/Fe₂O₃, 2.5% Au/C, 2.5% Pd/TiO₂, 2.5% Pd/Al₂O₃, 2.5% Pd/SiO₂, 2.5% Pd/Fe₂O₃, and 2.5% Pd/C.

49. The method of claim 48, wherein the allylic oxidation catalyst comprises (a) gold and/or palladium, and (b) titanium, aluminum, silicon, iron, and/or carbon.

50. The method of claim 48, wherein the allylic oxidation catalyst is free from chromium-based and copper-based oxidants.

51. The method of claim 48, wherein the allylic compound comprises a first compound selected from the group consisting of α-pinene, valencene, isophorone, and guaiene and wherein the allylic oxidation catalyst catalyzes an oxidation of the first compound to to form at least one oxidized version of the first compound.

52. The method of claim 51, wherein the at least one oxidized version of the first compound is verbenone if the first compound is α-pinene, nookatone if the first compound is valencene, 4-oxoisophorone is the first compound is isophorone, or rotundone if the first compound is guaiene.

53. The method of claim 48, wherein the allylic oxidation catalyst catalyzes an oxidation of the allylic compound to form at least one fragrance or one flavor compound.

54. The method of claim 48 further comprising the steps of:
introducing the allylic compound into a reaction chamber;
introducing the allylic oxidation catalyst into the reaction chamber;
purging the reaction chamber with oxygen; and
raising a temperature within the reaction chamber to facilitate an oxidation of the allylic compound using the allylic oxidation catalyst.

55. The method of claim 54, wherein the reaction chamber comprises a stainless steel autoclave.

56. The method of claim 54, wherein the allylic compound comprises a compound selected from the group consisting of α-pinene, valencene, isophorone, and guaiene.

57. The method of claim 54, wherein the allylic oxidation catalyst is free from chromium-based and copper-based oxidants.

58. The method of claim 54, wherein the step of purging the reaction chamber with oxygen comprises purging the reaction chamber using oxygen to leave the reaction chamber at a desired elevated pressure.

59. The method of claim 58, wherein the desired elevated pressure is selected from the group consisting of about 10 bar, about 20 bar, about 30 bar, between about 15 bar and about 25 bar, between about 25 bar and 35 bar, and greater than about 1 bar.

60. The method of claim 54, wherein the step of raising the temperature of the allylic compound and the allylic oxidation catalyst within the reaction chamber comprises raising the temperature to a level selected from the group consisting of at least about 50° C., at least about 60° C., at least about 75° C., and between about 40° C. and about 95° C.

61. The method of claim 54, wherein the method further comprises the step of:
stirring the allylic compound and the allylic oxidation catalyst within the reaction chamber prior to and/or during the step of raising the temperature of the allylic compound and the allylic oxidation catalyst within the reaction chamber to facilitate the oxidation of the allylic compound.

62. The method of claim 61, wherein the step of stirring the allylic compound and the allylic oxidation catalyst comprises stirring the allylic compound and the allylic oxidation catalyst within the reaction chamber at a speed between 100 rpm and 2500 rpm.

63. A system for oxidizing allylic compounds, the system comprising:
a reaction chamber for receiving at least one allylic compound and at least one allylic oxidation catalyst;
a gas source operably coupled to the reaction chamber, the gas source operable to introduce a gas into the reaction chamber to increase a pressure within the reaction chamber;
a heating source in conductive communication with the reaction chamber, the heating source operable to provide heat to the reaction chamber to increase a temperature within the reaction chamber;
a stirrer for stirring contents within the reaction chamber; and
an amount of an allylic oxidation catalyst placed within the reaction chamber,
the allylic oxidation catalyst comprising (a) gold and/or palladium, and (b) titanium, aluminum, silicon, iron, and/or carbon;
wherein the allylic oxidation catalyst catalyzes an oxidation of the allylic compound after the allylic compound is placed within the reaction chamber with the allylic oxidation catalyst, and wherein the oxidation of the allylic compound produces an oxidized allylic compound.

64. The system of claim 63, wherein the reaction chamber comprises a stainless steel autoclave.

65. The system of claim 63, wherein the gas source comprises a source of oxygen, and wherein the gas comprises oxygen.

66. The system of claim 63, wherein the allylic oxidation catalyst comprises 2.5% Au+2.5% Pd/TiO₂, 1.00% Au+1.0% Pd/TiO₂, 1.0% Au+2.0% Pd/TiO₂, 3.0% Au+3.0% Pd/TiO₂, 4.0% Au+4.0% Pd/TiO₂, 5.0% Au+5.0% Pd/TiO₂, 2.0% Au+3.0% Pd/TiO₂, 3.0% Au+2.0% Pd/TiO₂, 1.0% Au+4.0% Pd/TiO₂, 4.0% Au+1.0% Pd/TiO₂, 2.0% Au+2.5% Pd/TiO₂, 2.5% Au+2% Pd/TiO₂, 2.5% Au+2.5% Pd/Al₂O₃, 2.5% Au+2.5% Pd/SiO₂, 2.5% Au+2.5% Pd/Fe₂O₃, 2.5% Au+2.5% Pd/C, 2.5% Au/TiO₂, 2.5% Au/Al₂O₃, 2.5% Au/SiO₂, 2.5% Au/Fe₂O₃, 2.5% Au/C, 2.5% Pd/TiO₂, 2.5% Pd/Al₂O₃, 2.5% Pd/SiO₂, 2.5% Pd/Fe₂O₃, and 2.5% Pd/C.

67. The system of claim 63, wherein the allylic oxidation catalyst comprises palladium, gold, and titanium.

68. The system of claim 63, wherein the allylic oxidation catalyst is free from chromium-based and copper-based oxidants.

69. The system of claim 63, wherein the allylic compound is selected from the group consisting of α-pinene, valencene, isophorone, and guaiene, and wherein the oxidized allylic compound is selected from the group consisting of verbenone, nookatone, 4-oxoisophorone, and rotundone.

70. A method for catalyzing an allylic oxidation reaction, the method comprising the steps of
providing an allylic oxidation catalyst within a reaction chamber, the allylic oxidation catalyst comprising (a) gold and/or palladium, and (b) titanium, aluminum, silicon, iron, and/or carbon;
introducing an allylic compound into a reaction chamber;
increasing a pressure within the reaction chamber; increasing a temperature within the reaction chamber; stirring contents within the reaction chamber; and
cooling contents within the reaction chamber, wherein at least part of the cooled contents comprise an oxidized allylic compound.

71. An oxidized allylic compound, the oxidized allylic compound prepared by combining an allylic compound and an allylic oxidation catalyst comprising (a) gold and/or palladium, and (b) titanium, aluminum, silicon, iron, and/or carbon.

72. The oxidized allylic compound of claim 71, wherein the allylic compound comprises α-pinene, valencene, isophorone, or guaiene; and wherein the oxidized allylic compound comprises verbenone if the allylic compound is α-pinene, nookatone if the allylic compound is valencene, 4-oxoisophorone is the allylic compound is isophorone, or rotundone if the allylic compound is guaiene.
